(19) United States
(12) Patent Application Publication
(10) Pub. No.: US 2020/0189921 A1
(43) Pub. Date: Jun. 18, 2020

(54) MATRIX OF RICE HUSK SILICA FOR IMMOBILIZING ENZYME AND USES THEREOF

(71) Applicant: ICOSATECH INC., Jeollanam-do (KR)

(72) Inventors: Ok Soo HAN, Jeollanam-do (KR); Chong Soo HAN, Jeollanam-do (KR); Bao Thu LE, Jeollanam-do (KR); Putheary NGIN, Jeollanam-do (KR)

(21) Appl. No.: 16/617,998

(22) PCT Filed: Jan. 17, 2018
(86) PCT No.: PCT/KR2018/000812

§ 371 Date: Nov. 27, 2019

(30) Foreign Application Priority Data
May 31, 2017 (KR) 10-2017-0067384

(51) Int. Cl. C01B 33/18; C12N 11/14

(52) U.S. Cl. CPC C01B 33/18; C12Y 503/99006; C01P 2006/16; C12N 11/14

(57) ABSTRACT

The present invention relates to a preparation method of a matrix of rice husk silica for immobilizing enzyme wherein a rice husk silica is modified with APTES((3-aminopropyl)triethoxysilane) and glutaraldehyde, a matrix of rice husk silica for immobilizing enzyme prepared by said method, a rice husk silica-enzyme complex wherein enzyme is immobilized onto said matrix of rice husk silica and a preparation method of valuable substances using said rice husk silica-enzyme complex. The rice husk silica having a nanoporous structure cannot only facilitate diffusion of substrate during enzyme reaction but also can be utilized as a path through which intermediates from the consecutive enzyme reactions can travel. Therefore, it can be effectively utilized in preparing valuable substances.

### TECHNICAL FIELD

The present invention relates to a matrix of rice husk silica for immobilizing enzyme and uses thereof. The present invention, more specifically, relates to a preparation method of a matrix of rice husk silica for immobilizing enzyme wherein a rice husk silica is modified with APTES((3-aminopropyl)triethoxysilane) and glutaraldehyde, a matrix of rice husk silica for immobilizing enzyme prepared by said method, a rice husk silica-enzyme complex wherein enzyme is immobilized onto said matrix of rice husk silica, and a preparation method of valuable substances using said rice husk silica-enzyme complex.

### BACKGROUND

Rice, as a defensive respiratory system, has an amorphous silica layer formed in a rice husk covering rice grain. This layer has nano sized pores, and thus has a high permeability to gases and shows molecular flow characteristic. A rice Husk Silica (RHS) obtained from a rice husk also has a number of pores (˜4 nm) in silica particles, and thus is characterized by having a remarkably large surface area per unit mass (˜250 m²/g). The characteristic of rice husk silica mentioned above provides a significant benefit: the silica can be used in a solid state for immobilizing enzyme. Further, the nanoporous silica produced from a rice husk is natural silica which has low toxicity to human body, and high chemical activity compared with inorganic nano porous silica or mesoporous silica obtained from the reactions between aqueous silicate solution and inorganic acid, and thus facilitates modification and also can be easily molded in various shapes and mass-produced with low cost. Therefore, it can be considered as more effective material to immobilize proteins and biosynthetic enzymes. In particular, nanopores of a rice husk silica cannot only provide spatial sites for chemical immobilization of enzymes by a covalent bond so as to increase the immobilization efficiency but also can be used as effective moving passages for intermediates in a multi-step biosynthetic enzyme reaction system. Therefore, they can be used in the multi-step enzyme reaction.

Oxylipins are biologically active lipids derived and produced from fatty acids, which are used as important defensive materials in organisms. In spite of their significance, however, due to the structural complexity of oxylipin molecules including stereochemistry, their chemical synthesis for commercial preparing has not been generalized. In addition, various enzymes which are related to metabolism of oxylipins and represent biologically important function are present separately in some organelles in a cell. Under the circumstances, currently it is very difficult to produce oxylipins in vivo by controlling their biosynthetic paths.

The inventors of the present application had been focusing on developing a system for immobilizing enzyme in vitro for synthesis of oxylipins in order to overcome current problems in effective preparation of oxylipins, and then they eventually completed a commercial preparation method of oxylipins such as oxophytodienoic acids (OPDAs) through the immobilization of essential enzymes for biosynthesizing oxylipins, based on the idea that the nanoporous silica produced from a rice husk, a by-product of rice, is a natural silica which is the optimal material for immobilizing enzyme for biosynthesizing oxylipins.

On the other hand, the Korean Patent Registration No. 0396457 discloses ‘Method for preparing porous silica, porous silica-based molding material, and nano-sized silica particle derived from rice husk’, and the Korean Patent Publication No. 2013-0071451 discloses ‘Method for preparing high-purity silica derived from rice husk’. However, a matrix of rice husk silica for immobilizing enzyme and uses thereof are not disclosed at all.

### SUMMARY

### Technical Problem

The present invention is derived from the desire mentioned above. The inventors found that protein or enzyme could be immobilized in a high level as a result of immobilizing them with a rice husk silica having a nanoporous structure modified with APTES((3-aminopropyl)triethoxysilane) and glutaraldehyde. Further, they also found that immobilizing enzymes which are involved in oxylipin synthesis to said rice husk silica, and then followed by synthesizing oxylipin compounds using linolenic acids as substrate can result in oxophytodienoic acids, whereby the present invention has been accomplished.

### Solution to Solve the Problem

In order to solve the above-mentioned problem, the present invention provides a preparation method of a matrix of rice husk silica for immobilizing enzyme, comprising a) a step of mixing 18-24% of APTES((3-aminopropyl)triethoxysilane) to a rice husk silica (RHS) to react them at temperature between 38° C. and 42° C.; b) a step of recovering a rice husk silica modified with APTES (RHS-ATPES) by removing remaining unreacted APTES after centrifuging the reaction mixture of said step (a); c) a step of mixing glutaraldehyde to the rice husk silica modified with APTES (RHS-APTES) recovered from said step b) to react them at room temperature; and d) a step of recovering a rice husk silica modified with APTES and glutaraldehyde (RHS-APTES-GDA) by removing remaining unreacted glutaraldehyde after centrifuging the reaction mixture of said step (c).

Further, the present invention provides a matrix of rice husk silica for immobilizing enzyme prepared by said method.

Further, the present invention provides a rice husk silica-enzyme complex wherein enzyme is immobilized onto said matrix of rice husk silica.

Further, the present invention provides a preparation method of valuable substances using said rice husk silica-enzyme complex.

### Advantageous Effects of Invention

According to the present invention, the nanoporous structure of a rice husk silica cannot only facilitate diffusion of substrate during an enzyme reaction but also can be utilized as a path through which intermediates from the consecutive enzyme reactions can travel, and therefore, it can be effectively utilized advantageously in preparing biosynthetic products. In addition, the present invention can provide an opportunity to raise a rice husk, a by-product of rice, in value, and it can be useful for immobilizing biosynthetic enzymes involved in metabolism of fatty acids, such as the preparation of oxylipins using fatty acids, which is hydrophobic molecules, as substrate, and for mass preparing of metabolites using the same.

### BRIEF DESCRIPTION OF DRAWINGS

FIG. 1 depicts a structure of the product resulting from immobilization of enzyme (protein) onto the rice husk matrix with APTES((3-aminopropyl)triethoxysilane) and glutaraldehyde (GPA).

FIG. 2 depicts delivery of biosynthetic intermediates through nanopores of a rice husk silica, and an application method thereof to multi-step enzyme reactions.

FIG. 3 sequentially depicts a procedure for preparing 12-oxophytodienoic acid (OPDA) by way of consecutive reactions with a lipoxygenase (LOX), an allene oxide synthase (AOS) and an allene oxide cyclase (AOC), in a multi-step path for biosynthesizing oxylipin, covering from linolenic acid to jasmonic acid.

FIG. 4 shows HPLC analysis results of OPDA resulting from consecutive reactions of linolenic acid with LOX, AOS and AOC, including SP-HPLC analysis (A: AOS reaction, B: AOS/AOC reaction) and RP-HPLC analysis (C: AOS reaction, D: AOS/AOC reaction) results of reaction products using 13(S)-hydroperoxides of α-linolenic acid (13-HPOT) resulting from LOX. CP-HPLC results of cis-OPDAs (12-OPDAs) prepared by AOS reaction and AOS/AOC reaction are inserted into A and B, respectively.

FIG. 5 shows the amount of hydroperoxyoctadecatrienoic acid (HPOT), the product resulting from enzyme reactions depending on the amount of lipoxygenase used, for the rice husk silica with free soybean LOX (A) and for the rice husk silica with soybean LOX immobilized thereon (B), respectively.

FIG. 6 shows the result demonstrating the amount of products resulting from each enzyme reaction according to the number of times of recycling of each immobilization system prepared by immobilizing LOX, AOS or AOC onto a rice husk silica with APTES and GDA, is recycled.

FIG. 7 shows the result demonstrating the amount of OPDA of co-immobilization using GDA (GDA-(LOX-AOS-AOC)) and co-immobilization using epichlorohydrin (ECH) and PEG (ECH-PEG-(LOX-AOS-AOC)), in the method for the synthesis of cis-OPDA by successive sequential reactions of LOX, AOS and AOC onto rice husk silica.

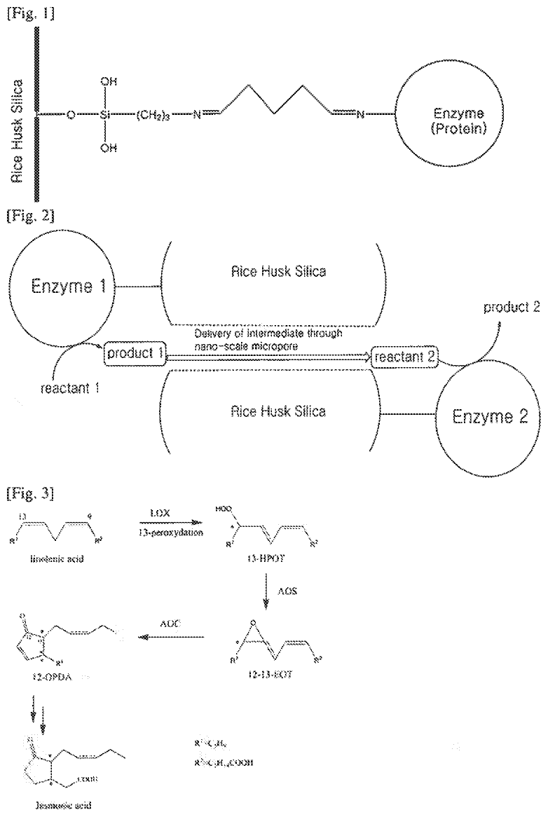

### MODE FOR THE INVENTION

The present invention provides a method of preparing a matrix of rice husk silica for immobilizing enzyme to achieve the purpose thereof.

In one embodiment of the present invention, a method of preparing a matrix of rice husk silica for immobilizing enzyme comprises, specifically,

a) a step of mixing 18-24% of APTES((3-aminopropyl)triethoxysilane) to a rice husk silica (RHS) to react them at temperature between 38° C. and 42° C. for 1.5˜2.5 hours;

b) a step of recovering a rice husk silica modified with APTES (RHS-ATPES) by removing remaining unreacted APTES after centrifuging the reaction mixture of said step (a);

c) a step of mixing glutaraldehyde (GDA) to the rice husk silica modified with APTES (RHS-APTES) recovered from said step b) to react them at room temperature; and

d) a step of recovering a rice husk silica modified with APTES and glutaraldehyde (RHS-APTES-GDA) by removing remaining unreacted glutaraldehyde after centrifuging the reaction mixture of said step (c), and more specifically, it may comprise, but not limited to,

a) a step of mixing 21% of APTES to a rice husk silica (RHS) to react them at temperature of 40° C. for 2 hours;

b) a step of recovering a rice husk silica modified with APTES (RHS-APTES) by removing remaining unreacted glutaraldehyde after centrifuging the reaction mixture of said step (a);

c) a step of mixing glutaraldehyde (GDA) to the APTES-modified rice husk silica recovered from said step b) to react them at room temperature for 4 hours; and

d) a step of recovering a rice husk silica modified with APTES and glutaraldehyde (RHS-APTES-GDA) by removing remaining unreacted glutaraldehyde after centrifuging the reaction mixture of said step (c).

In the matrix of rice husk silica for immobilizing enzyme of the present invention, said rice husk silica not only functions as a solid phase support but also facilitates diffusion of substrate during enzyme reactions through its nanoporous structure. Further, it can be utilized as a path through which intermediates from the consecutive enzyme reactions can travel; and therefore, is an effective material for preparing synthetized products.

In the technical field of immobilizing biomolecules (for example, enzyme protein), a main technical problem to be solved is to bond a high density of biomolecules to a small area and to preserve physiological active function of the bonded biomolecules to the maximum. Further, the immobilization technology needs to be performed easily and simply by anyone with cost effectiveness, which is a requirement to conduct research and development for the physiological active substance more effectively.

The present invention also provides a matrix of rice husk silica for immobilizing enzyme prepared by said method.

The matrix of rice husk silica for immobilizing enzyme of the present invention can lead to preparing cost reduction with use of a rice husk, a by-product of rice, and a spacer material modified onto the surface of rice husk silica and am enzyme to be immobilized can be easily and simply immobilized via a covalent bond between them. Therefore, it can be recognized as an immobilization technology of enzyme (or biomaterials) having a good industrial applicability.

The present invention also provides a rice husk silica-enzyme complex wherein enzyme is immobilized onto said matrix of rice husk silica and a preparation method of valuable substances using said rice husk silica-enzyme complex.

Said rice husk silica-enzyme complex of the present invention in which an active enzyme to produce the desired valuable substances is immobilized onto the above-mentioned matrix of rice husk silica for immobilizing enzyme, can have at least one or two enzyme(s) immobilized therein, which is (are) involved in the biosynthesis process of the desired valuable substances.

In the rice husk silica-enzyme complex according to one embodiment of the present invention, said enzyme can be, but is not limited to, at least one selected from the group consisting of a lipoxygenase (LOX), an allene oxide synthase (AOS) and an allene oxide cyclase (AOC). Said LOX, AOS and AOC are enzymes involved in the multi-step biosynthesis of oxylipin, and said enzymes can produce oxophytodienoic acid (OPDA) or jasmonic acid derivatives, etc., using linolenic acid as substrate, which can be used as pest control agents and anticancer substances.

In the method for preparing valuable substances according to one embodiment of the present invention, said valuable substances can be, but is not limited to, oxylipin compounds. The method for preparing valuable substances of the present invention provides a system that can simply produce in large quantities of valuable substances through immobilization of useful enzymes onto rice husk silica having nanoporous structure with a covalent bond. In particular, when preparing valuable substances requiring a multi-step biosynthetic process, the nanoporous structure of a rice husk silica can facilitate diffusion and travel of substrate and/or intermediates and improve production efficiency of the valuable substances.

The invention will be further described through the following examples below. The following examples are set forth to illustrate, but are not to be construed to limit the present invention.

### Example 1. Production of an Allene Oxide Synthase and an Allene Oxide Cyclase for Chemical Immobilization

To produce an allene oxide synthase (AOS), an allene oxide synthase (AOS) gene of rice (OsAOS1) was incorporated into a pET28b vector to prepare a pET28b-OsAOS1 vector for heterologous expression of OsAOS1 enzyme, to transform E. coli BL21(DE3) into a pET28b-OsAOS1 vector, to use the transformed E. coli BL21(DE3) as a seed culture and incubate it at 37° C. for 2 hours, then to induce OsAOS1 protein expression using IPTG, and then to culture for additional 6 hours. From the cell paste resulting from centrifugation, cells with OsAOS1 protein expressed were disrupted by using sonication buffer (50U DNase, 0.2 mM PMSF, 50 mM sodium phosphate, pH 7.5) containing 5 mM Emulphogene. The resulting cell lysate was centrifuged to remove cell debris, and the supernatant containing OsAOS1 protein was purified using a Q-Sepharose column.

To produce an allene oxide cyclase (AOC), an allene oxide cyclase (AOC) gene of rice (OsAOC) was incorporated into a pRSETB vector to prepare pRSETB-OsAOC vectors, to incubate the transformed E. coli BL21(DE3)pLysS using said vector as a seed culture at 37° C. for 4-5 hours, then to induce OsAOC protein expression using IPTG, and then to culture for additional 6 hours. Then, the cell paste resulting from centrifugation was suspended in 50 mM sodium phosphate buffer (pH 7.5) containing 0.2% Tween 20 and 10 mM EDTA. To this, 0.2 mM of phenylmethane sulfonyl fluoride (PMSF) was added, and then followed by cell disruption using sonication. The resulting cell lysate was centrifuged to remove cell debris, and protein precipitates containing OsAOC protein were obtained by adding 40% ammonium sulfate to the supernatant containing OsAOC protein. Ammonium sulfate was removed via dialysis and then followed by purification using Q-Sepharose column.

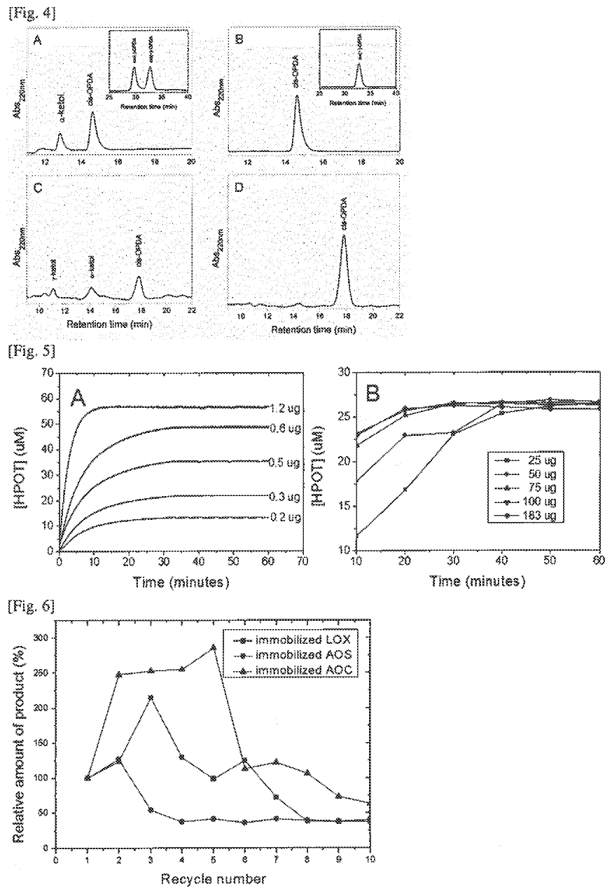

BSA (bovine serum albumin) and soybean lipoxygenase (LOX) were purchased from Sigma-Aldrich (US) and used. Activities of LOX and AOS were measured by a known method (Yoeun et al. 2013, BMB reports 46:151), and OPDA (oxophytodienoic acid) was synthesized from linolenic acid via connecting reactions of LOX, AOS and AOC (FIG. 3). Linolenic acid was used with LOX to synthesize HPOT (hydroperoxides of α-linolenic acid) to which AOS and AOC were subsequently reacted to provide the reaction products, and then the resulting products were extracted with dichloromethane and separated with straight phase HPLC (SP-HPLC) to collect cis-OPDA fractions (FIGS. 4A and 4B). For the collected cis-OPDA, the structure was identified via GC/MS analysis, and the ratio of cis(+)-OPDA and cis(−)-OPDA in the resulting cis-OPDA was analyzed with chiral phase HPLC (CP-HPLC) (Drawings inserted in FIGS. 4A and 4B). Further, OPDA synthesis efficiency was analyzed using reversed phase HPLC(RP-HPLC) which can directly analyze it without extracting LOX/AOS/AOC reaction products (FIGS. 4C and 4D). The wavelengths from a UV-detector used for α-ketol, OPDA and HPOT during HPLC analysis were 205 nm, 220 nm and 234 nm, respectively. From the result of said analysis, it was confirmed that cis(+)-OPDA could be effectively produced from linolenic acid using LOX derived from soybean, and AOS and AOC enzymes from rice.

### Example 2. Immobilization of Protein Using a Rice Husk Silica (RHS) Having Nanopores

According to the method provided in the Korean Patent Registration No. 0396457, a rice husk silica having an average pore diameter of 50-500 nm and canals of 10 nm or less was prepared through the process of acid treatment-carbonization-acid treatment-oxidation. 100 mg of the prepared rice husk silica (RHS) was mixed with 21% (w/v) APTES ((3-aminopropyl)triethoxysilane) diluted in ethyl alcohol, then followed by gentle shaking at 40° C. for two hours. The mixture was centrifuged and washed with ethyl alcohol and 50 mM sodium phosphate buffer (pH 7.2), respectively to remove the remaining APTES. Then, a rice husk silica modified with APTES (RHS-APTES) was obtained. 1% Glutaraldehyde (GDA) was added to RHS-APTES (10 mg) re-suspended in 1 ml of 50 mM sodium phosphate buffer (pH 7.2), then followed by gently shaking at room temperature for 4 hours to produce pale pink products. The resulting products were centrifuged to obtain a rice husk silica with glutaraldehyde (RHS-APTES-GDA). The remaining GDA was washed with 50 mM sodium phosphate buffer (pH 7.2). Then, about 300 μg of BSA was added, and followed by reaction at room temperature for 24 hours. After centrifugation, the resulting products were washed with a solution comprising 1M NaCl and 1% Tween 20 to completely remove the non-immobilized proteins. Then, a composition having a rice husk silica with BSA immobilized thereon via covalent bonds (RHS-APTES-GDA-BSA) was obtained. For the chemical immobilization of a lipoxygenase (LOX), an allene oxide synthase (AOS) and an allene oxide cyclase (AOC), a lipoxygenase was purchased from Sigma-Aldrich, and an allene oxide synthase (OsAOS1) and an allene oxide cyclase (OsAOC) of rice were prepared according to the method described in said Example 1. They were reacted with RHS-APTES-GDA in a similar way to BSA to obtain RHS-APTES-GDA-LOX, RHS-APTES-GDA-AOS, and RHS-APTES-GDA-AOC compositions, respectively, in which each enzyme was immobilized to a rice husk silica via a chemical covalent bond. The amount of the immobilized protein in each protein immobilization was calculated to analyze the immobilization efficiency (Table 1). The immobilization efficiency varied depending on the type of proteins, showing about 50-93%.

TABLE 1
Chemical immobilization efficiency of proteins using
a rice husk nanoporous silica as a matrix^{a}

| | | Amount of non- | Amount of | |
|---|---|---|---|---|
| | Amount of | immobilized | immobilized | Immobilization |
| | Protein | protein | protein | efficiency |
| Protein | (A) (μg) | (B)^{b} (μg) | (C = A − B) (μg) | ((C/A) × 100) (%) |
| BSA | 320.3 ± 0.9 | 108.0 ± 2.0 | 211.6 ± 2.0 | 66.2 ± 0.6 |
| LOX | 251.2 ± 0.9 | 69.4 ± 5.7 | 191.4 ± 5.7 | 73.4 ± 2.2 |
| AOS^{c} | 288.1 ± 0.9 | 145.1 ± 3.5 | 143.1 ± 3.5 | 49.6 ± 1.2 |
| AOC | 194.7 ± 0.9 | 14.7 ± 0.4 | 180.0 ± 0.4 | 92.5 ± 0.2 |

^{a}The experimental results are shown as ‘the mean value ± the standard deviation’ obtained by repeating the experiment three times.
^{b}Concentration of the non-immobilized proteins remaining in the solution is quantitatively determined by the bicinchoninic acid (BCA) assay.
^{c}including 0.02% Emulphogene

### Example 3. Analysis of Activities of the Enzymes Immobilized to a Rice Husk Nanoporous Silica (RHS)

The activity of LOX immobilized onto a rice husk silica was measured using the xylenol orange assay (del Carmen Pinto et al., (2007) J. Agric. Food Chem. 55:5956-5959). The activity of AOS was measured using HPOT prepared with LOX as substrate (Yoeun et al., (2013) BMB Reports 2013; 46: 151-156), and then connecting reactions of LOX, AOS, and AOC from linolenic acid synthesized OPDA. The activity of AOC was shown as the amount of the produced OPDA which was separated using RP-HPLC. The result of comparing the activity of immobilized enzymes to that of non-immobilized (free) enzymes (Table 2), it was acknowledged that the activities of LOX and AOS significantly reduced by the immobilization of enzymes using the covalent bonding method, while the one of AOC slightly increased.

TABLE 2
Comparison of activities of enzymes in the immobilized
system and non-immobilized (free) system

| | specific activity | | |
|---|---|---|---|
| | (μmol/sec · μg) | | Relative Activity |
| Enzyme | Free System | Immobilized System | (%)^{a} |
| LOX | 1.1 × 10⁻⁵ | 2.7 × 10⁻⁷ | 2.4 |
| AOS | 3.8 × 10⁻⁴ | 4.8 × 10⁻⁶ | 1.3 |
| AOC^{b} | 464.7 | 507.2 | 109.0 |

^{a}Relative catalytic efficiency of the immobilized system for non-immobilized (free) system, expressed as a percentage
^{b}Relative amount of OPDA prepared by AOC reaction was determined with RP-HPLC Chromatogram, expressed as a percentage

### Example 4. Analysis of the Amount of Products for the Amount of Enzyme Used in the LOX Enzyme Reaction

In case of a lipoxygenase, the result of analysis of the amount of HPOT, the product resulting from enzyme reactions over the reaction time shows that the amount of the produced HPOT reduced as the total amount of enzymes used reduced, in free LOX. Such result indicates that in case of free LOX, inactivation occurs, as the enzyme reaction proceeds. However, the amount of HPOT produced was kept constant, regardless of the amount of enzymes used in immobilized LOX (FIG. 5). Accordingly, although immobilization of said enzyme with a rice husk silica and GDA decreased the specific activity of LOX enzyme (Table 2), it could prevent LOX enzyme from being inactivated over the reaction (FIG. 5). Thus, it can be considered as an effective method to produce HPOT.

### Example 5. Change in Activity after Recycling the Immobilized Enzyme

In order to evaluate durability of an immobilized enzyme system, APTES and GDA were used to immobilize LOX, AOS and AOC, respectively, and the amount of product resulting from the enzyme reactions after recycling each immobilization system was analyzed as shown in Example 3. Consequently, for the LOX immobilization system, it was confirmed that the amount of products resulting from enzyme reactions increased until three cycles of recycling, but significantly decreased at the fourth, and since then said amount gradually decreased. In case of the AOS immobilization system, it was confirmed that the amount of products resulting from enzyme reactions increased until two cycles of recycling, but significantly decreased from the third cycle. In case of the AOC immobilization system, it was confirmed that the amount of products resulting from enzyme reactions increased until five cycles of recycling, but significantly decreased from the sixth cycle. Specifically, in case of AOC, it was confirmed that the amount of products was more than for LOX or AOS enzyme on the basis of the same cycles of recycling (FIG. 6). It was thought to be attributed to the high immobilization efficiency of AOC (Table 1). It was considered that the reason why the amount of the reaction product after recycling increased was because the product from the enzyme reaction after the first cycle was adsorbed to a rice husk silica and was not precipitated into the solution.

### Example 6. Co-immobilization system of a lipoxygenase, an allene oxide synthase and an allene oxide cyclase

The results of preparing OPDA using the followings were compared to each other: Immobilization system prepared using glutaraldehyde (GDA) with linolenic acid as a starting material (GDA-(LOX-AOS-AOC)) and Immobilization system prepared using ECH-PEG as a spacer (ECH-PEG-(LOX-AOS-AOC)).

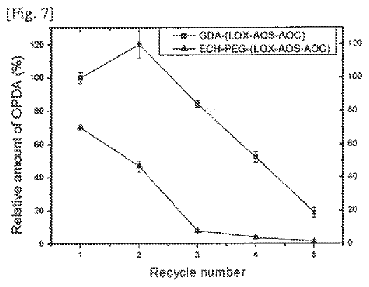

Consequently, it was confirmed that the co-immobilization system (GDA-(LOX-AOS-AOC)) had a higher efficiency of preparing OPDA than the co-immobilization system, ECH-PEG-(LOX-AOS-AOC), and also a high durability after recycling (FIG. 7).

1. A method of preparing a matrix of rice husk silica for immobilizing an allene oxide cyclase, said method comprising:
a) mixing 18-24% of APTES((3-aminopropyl)triethoxysilane) with a rice husk silica (RHS) to react with each other at a temperature between 38° C. and 42° C.;
b) recovering a rice husk silica modified with APTES (RHS-ATPES) by removing remaining unreacted APTES after centrifuging the reaction mixture of said step (a);
c) mixing glutaraldehyde (GDA) to with the rice husk silica modified with APTES (RHS-APTES) recovered from said step b) to react with each other at room temperature; and
d) recovering a rice husk silica modified with APTES and glutaraldehyde (RHS-APTES-GDA) by removing remaining unreacted glutaraldehyde after centrifuging the reaction mixture of said step (c).

2. The method according to claim 1, characterized in that said rice husk silica has a nanoporous structure.

3. A matrix of rice husk silica for immobilizing an allene oxide cyclase prepared by the method according to claim 1.

4. A rice husk silica allene oxide cyclase complex wherein an allene oxide cyclase is immobilized onto the matrix of rice husk silica according to claim 3.

5. (canceled)

6. A process for the preparation of valuable substances using the rice husk silica allene oxide cyclase complex having immobilized allene oxide cyclase therein according to claim 4.

7. The process for the preparation of valuable substances according to claim 6, wherein said valuable substances are oxylipin compounds.

8. The process for the preparation of oxophytodienoic acid (OPDA) according to claim 6, using the rice husk silica allene oxide cyclase complex wherein a lipoxygenase, an allene oxide synthase and an allene oxide cyclase are immobilized therein with linolenic acid as substrate.
